(19) United States
(12) Patent Application Publication
(10) Pub. No.: US 2019/0151586 A1
(43) Pub. Date: May 23, 2019

(54) TELESCOPIC ENDOTRACHEAL INTUBATION BOUGIE

(71) Applicant: Evan Denis Schmitz, La Jolla, CA (US)

(72) Inventor: Evan Denis Schmitz, La Jolla, CA (US)

(21) Appl. No.: 16/197,092

(22) Filed: Nov. 20, 2018

Related U.S. Application Data
(60) Provisional application No. 62/590,329, filed on Nov. 23, 2017.

(51) Int. Cl. A61M 16/04

(52) U.S. Cl. CPC A61M 16/0402; A61M 16/0434; A61M 16/0488

(57) ABSTRACT

A device for opening an intubating a constricted airway, the device having a first elongated element and a rigid body with a longitudinal axial bore that is formed of non-malleable, preferably unbendable material, a rigid rod having a distal end and a proximal end, the rod having a body formed of non-malleable, unbendable material and sized and shaped to be slidably received within the longitudinal axial bore of the first rigid elongated element to move between a collapsed position in which the rigid rod is slidably received within the body first elongated element and an extended position in which the rod slidably extends out of the first elongated element, a locking mechanism that permanently holds the rod in the extended position within the first elongated element, a rigid tip formed on the distal end of the rod, the tip having a smooth, spherical terminal end.

### BACKGROUND

### Technical Field

The present disclosure pertains to devices for insertion into constricted airways and, more particularly, to an intubation bougie to be used in opening a constricted airway as well as for guiding a tube through a constricted airway.

### Description of the Related Art

Intubation is the process of inserting a tube into an airway. In difficult breathing situations, time is of the essence in opening the airway and placing the tube. Orotracheal intubation with a tube and stylet has been shown to be inconsistent in effectiveness in difficult airway situations, such as fluid obscuring the laryngeal view, airway obstruction or edema, obesity, short neck, small mandible, large tongue, facial trauma, or the need for cervical spine immobilization.

Current bougies and other modern tools for visualizing the glottis are at times inadequate. Even when the glottis is visualized, sometimes it is impossible to manipulate the endotracheal tube enough to maneuver it past the vocal cords and into the trachea. Failure to pass the endotracheal tube into the trachea can lead to death from respiratory failure in minutes. Despite the advances in video laryngoscopy and stylet technology there remains a need for a tool to ensure consistently successful endotracheal intubation in the critically ill patient.

In addition, the current technology has limitations in the treatment of acute respiratory failure where seconds can mean the difference between life or death. Currently there are elastic bougies in the market, but they are too flexible and are often bent into a storage position that compromises their integrity, which can lead to the inability for successful intubation causing death. Also, in an emergency situation these elastic bougies are difficult to locate in the field and in the hospital during a code blue situation where immediate action is required.

### BRIEF SUMMARY

The present disclosure is directed to an inflexible, rigid telescopic bougie that can fit in an emergency provider's scrub pocket, be expanded quickly, and be introduced into the airway in a patient with acute respiratory failure. The device can be sterilized and expanded in haste when needed, saving precious seconds, while attempting to intubate a patient with a difficult airway. The non-malleable bougie is able to overcome the compressive force of the oropharyngeal tissue, which not only inhibit the view of the vocal cords but can also block passage of a malleable stylet or elastic bougie, making intubation impossible and leading to death from acute respiratory failure.

In accordance with another aspect of the present disclosure, a device is provided that includes (a) a first elongated element having a distal end and a proximal end, and a rigid body with a longitudinal axial bore that is formed of non-malleable, unbendable material, (b) a second elongated element having a distal end and a proximal end, and a rigid body with a longitudinal axial bore that is formed of non-malleable, unbendable material, the first elongated element sized and shaped to be received in the longitudinal axial bore of the second elongated element and to move within the longitudinal axial bore between a collapsed position in which the first elongated element is slidably received within the longitudinal axial bore of the second elongated element and an extended position in which the first elongated element slidably extends out of the second elongated element, (c) a locking mechanism that holds the first elongated element in the extended position within the second elongated element, (d) a rigid rod having a distal end and a proximal end, the rod having a body formed of non-malleable, unbendable material and sized and shaped to be received within the longitudinal axial bore of the first rigid elongated element, and (e) a rigid tip formed on the distal end of the rod, the tip having a smooth, spherical terminal end and structured to project out of the first elongated element in either the collapsed or the extending position. In accordance with a further aspect of the present disclosure, the device includes the first rigid elongated element having a geometric cross-sectional configuration of one from among a square, a triangle, a rectangle, a circle, an oval, and a diamond and the locking mechanism permanently locking the first elongated element in the extended position so that it cannot be returned to the collapsed position.

In accordance with a further aspect of the present disclosure, the device includes a third elongated element formed of non-malleable, unbendable material and structured to slidably receive the second elongated element and a sleeve positioned between the rod and the longitudinal axial bore of the first elongated member, the sleeve, the rod, and the longitudinal axial bore of the first elongated member permanently coupled in a crimp fit.

In accordance with still yet a further aspect of the present disclosure, the locking mechanism comprises a second locking mechanism structured to permanently lock the second elongated element in an extended position relative to the third elongated element and a portion of the rigid rod proximate the rigid tip is at an angle to horizontal that is approximately 20 degrees.

In accordance with an alternative aspect of the present disclosure, a device is provided that includes (i) a first elongated element having an inflexible body between opposing distal ends and a longitudinal axial bore extending through the body between the opposing terminal ends, (ii) an inflexible rod received in the longitudinal axial bore of the first elongated element and coupled to the longitudinal axial bore of the first elongated element, the inflexible rod having a first portion proximate the first elongated element and a second portion separated from the first portion by a transition portion, wherein the first portion is at angle to the second portion, and (iii) a rigid tip coupled to and extending from the second portion, the tip having a smooth, spherical terminal end spaced from a distal end of the first elongated element.

In accordance with another aspect of the present disclosure, the angle between the first and second portions is between 10 and 30 degrees, or more preferably is approximately 20 degrees. Further, the first elongated member has a geometric cross-sectional configuration of one from among a square, a triangle, a rectangle, a circle, an oval, and a diamond.

In accordance with a further aspect of the present disclosure, the device includes a second elongated element having a longitudinal bore, the first elongated element telescopically received in the longitudinal bore of the second elongated element and the first elongated element slidable along the longitudinal bore of the second elongated element between a collapsed configuration and an extended configuration. The device further includes a locking mechanism that is structured to permanently lock the first elongated member in the extended configuration.

In yet another aspect of the present disclosure, the device further includes a third elongated element with a longitudinal bore, the second elongated element telescopically received in the longitudinal bore of the third elongated element, wherein the second elongated element is slidable along the longitudinal bore of the third elongated element between a collapsed configuration and an elongated configuration and a locking mechanism structured to permanently lock the second elongated member in the extended configuration.

### BRIEF DESCRIPTION OF THE SEVERAL VIEWS OF THE DRAWINGS

The foregoing and other features and advantages of the present disclosure will be more readily appreciated as the same become better understood from the following detailed description when taken in conjunction with the following drawings, wherein:

FIG. 1 is cross sectional view of a telescopic bougie formed in accordance with the present disclosure and illustrated in a collapsed position.

FIG. 2 is a cross sectional view of the telescopic bougie of FIG. 1 in an extended position.

FIGS. 3A-3B are axonometric views of locking mechanisms of the telescopic bougie of FIG. 2.

FIG. 4A is an axonometric view of the telescopic bougie of FIG. 1 with an endotracheal intubation tube positioned to slide over the telescopic bougie.

FIG. 4B is an axonometric view of the telescopic bougie of FIG. 2 with an endotracheal intubation tube positioned to slide over the telescopic bougie.

FIG. 5 is an axonometric view of the telescopic bougie of FIG. 2 illustrating a use condition where the bougie is inserted through a patient's vocal cords.

FIG. 6 is an axonometric view of the telescopic bougie of FIG. 2 illustrating a use condition where the bougie is inserted through a patient's vocal cords and an endotracheal intubation tube is slid over the bougie and past the vocal cords to provide air to a patient.

FIG. 7 is an axonometric view of the telescopic bougie of FIG. 1 illustrating a use condition where the bougie is used as a stylet and inserted through a patient's vocal cords and an endotracheal intubation tube is slid over the bougie and past the vocal cords to provide air to a patient.

### DETAILED DESCRIPTION

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures or components or both associated with endotracheal tubes as well as the process of intubation have not been shown or described in order to avoid unnecessarily obscuring descriptions of the implementations.

Unless the context requires otherwise, throughout the specification and claims that follow, the word “comprise” and variations thereof, such as “comprises” and “comprising” are to be construed in an open inclusive sense, that is, as “including, but not limited to.” The foregoing applies equally to the words “including” and “having.”

Reference throughout this description to “one implementation” or “an implementation” means that a particular feature, structure, or characteristic described in connection with the implementation is included in at least one implementation. Thus, the appearance of the phrases “in one implementation” or “in an implementation” in various places throughout the specification are not necessarily all referring to the same implementation. Furthermore, the particular features, structures, or characteristics may be combined in any suitable manner in one or more implementations. Reference throughout this description to “non-malleable” means that a particular feature, structure, or characteristic described as being non-malleable would break or crack if hammered or pressed permanently out of shape. Reference throughout this description to “unbendable” means that a particular feature, structure, or characteristic described as being unbendable is not able to be bent or is stiff. Reference throughout this description to “rigid” means that a particular feature, structure, or characteristic described as being rigid is not able to bend or be forced out of shape or is not flexible.

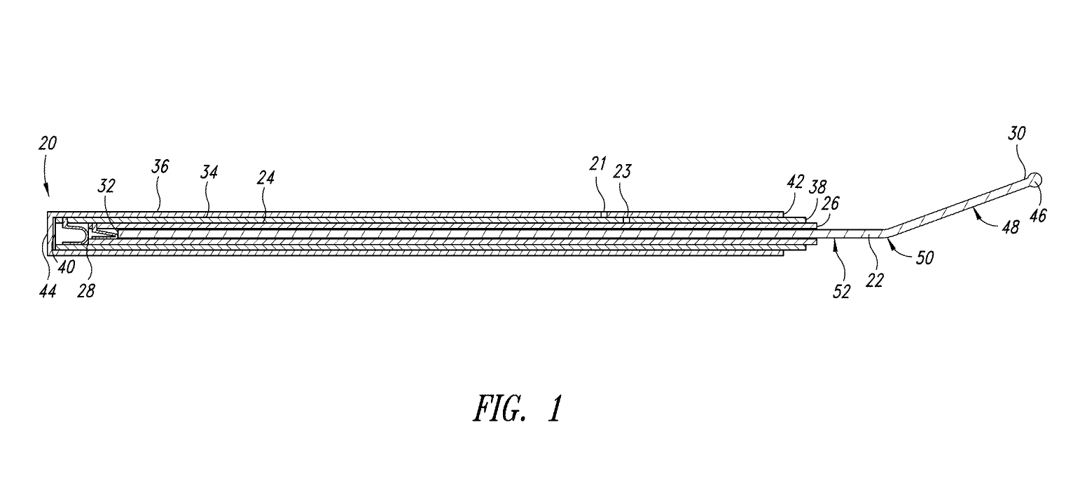

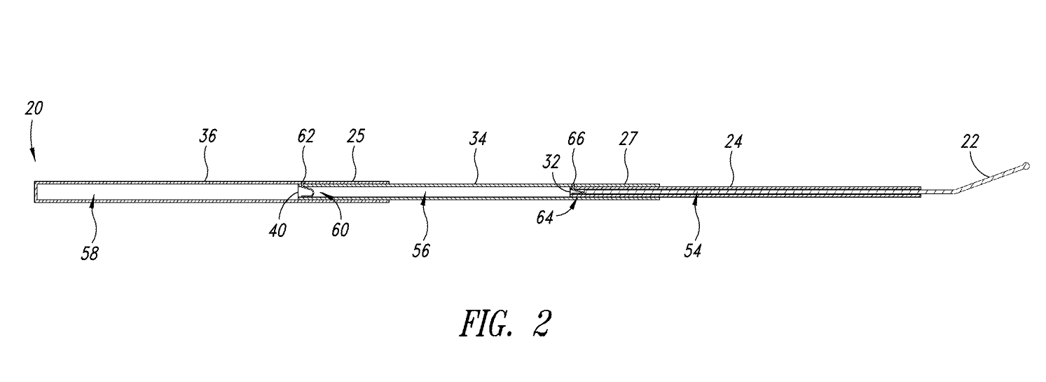

Referring initially to FIG. 1, illustrated therein is a telescopic bougie 20 including a rigid rod 22 coupled to a first tubular or elongated member 24 with the first elongated element 24 having a distal end 26 and a proximal end 28 illustrated in a collapsed position or configuration. As will be described herein, in the collapsed position or configuration, the bougie 20 may be referred to and used as a stylet. The first elongated element 24 has a rigid body which is preferably formed of non-malleable and unbendable material. In an implementation, the material is stainless steel, although other materials are suitable, for example, various other metals (i.e. aluminum) or surgical grade plastics. Such materials are capable of sterilization through one of various known methods, such that the bougie 20 can be sterilized prior to use. The first elongated element 24 has a longitudinal axial bore that will be described with reference to FIG. 2. In various implementations, the first elongated element 24 has a geometric cross-sectional configuration of one from among a square, a triangle, a rectangle, a circle, an oval, and a diamond.

The rigid rod 22 also includes a distal end 30 and a proximal end 32 and is similarly formed from non-malleable, unbendable material. In an implementation, the rigid rod 22 has a size and a shape to be received within the longitudinal axial bore of the first elongated element 24, as illustrated in FIG. 1. Although not immediately apparent in FIG. 1 due to the scale, there is a small gap or space between an outermost edge of the rigid rod 22 and an inner edge of the longitudinal axial bore of the first elongated element 24 that runs along a length of the rigid rod 22 that is received in the longitudinal axial bore of the first elongated element 24. A sleeve (not specifically shown due to scale), which may similarly be made of various metals, is positioned between the outermost edge of the rigid rod 22 and the longitudinal bore of the first elongated element 24 proximate the distal end 26 of the first elongated element 24.

The rigid rod 22 is preferably permanently coupled to the first elongated element 24 by crimping the first elongated element 24, the sleeve (not shown), and the rigid rod 22 together. Moreover, because the rigid rod 22 extends along almost an entire length (i.e. at least 80% of the length of the longitudinal axial bore of the first elongated element 24, but more preferably at least 90% of the length of the longitudinal axial bore of the first elongated element 24), the rigid rod 22 provides additional strength to the first elongated element 24 to prevent the first elongated element 24 from bending during use. It is to be further appreciated that other coupling methods may be used as an alternative to, or in addition to, crimping, such as various adhesives, welding, or other mechanical fasteners. However, in other implementations, the rigid rod 22 may be slidably received in the longitudinal axial bore of the first elongated element 24, similar to how the first elongated element 24 is slidably or telescopically received in a second tubular or elongated member, as described herein. In such implementations, an additional locking member may be used to permanently lock the rigid rod 22 in the extended configuration, as described herein.

FIG. 1 further illustrates that in some implementations, the bougie 20 includes a second tubular or elongated member 34 and a third tubular or elongated member 36. The second and third elongated elements 34, 36 can be similar to the first elongated element 24 (i.e. each may have a distal and proximal end and the same cross-sectional configuration). For example, the second elongated element 34 includes a distal end 38, a proximal end 40, a longitudinal bore (see FIG. 2) and may have the same cross sectional configuration as the first elongated element 24. Preferably, the first elongated element 24 has a size and a shape to be slidably received by the second elongated element 34 (i.e. the first elongated element 24 is movable between an extended configuration or position and a collapsed configuration or position relative to the second elongated element 34) such that the first elongated element 24 is telescopically received within the second elongated element 34, and more preferably, within the longitudinal axial bore of the second elongated element 34. Similarly, the third elongated element 36 includes a distal end 42, a proximal end 44, a longitudinal bore (see FIG. 2), and may have the same cross-sectional configuration as the first and second elongated elements 24, 34, wherein the second elongated element 34 has a size and a shape to be slidably received in a telescopic fashion by the third elongated element 36.

The bougie 20 further includes a rigid tip 46 formed on and extending from the distal end 30 of the rod 22. The tip 46 preferably has a smooth, spherical terminal end and is structured to project out of, and be spaced from, the first elongated element 24. In other words, in implementations where the rigid rod 22 is permanently affixed to the first elongated element 24, the tip 46 is spaced from the distal end 26 of the first elongated element. The rod 22 further includes a first portion 48 proximate the distal end 30 of the rod and a second portion 52 proximate the distal end of the first elongated member 24, wherein the first portion 48 is separated from the second portion 52 by a transition portion 50. Preferably, each of the first and second portions 48, 52 are straight and elongated with a continuous cross-sectional configuration and thickness or diameter along their length. The transition portion 50 is preferably rounded, such that the first portion 48 is positioned at an angle to the second portion 52. The angle between the portions 48, 52 is preferably between 10 and 30 degrees, and more preferably is 20 degrees or approximately 20 degrees. In this context only, “approximately” means within 2 degrees of 20 degrees (i.e. from 18 to 22 degrees). As such, when the first portion 48 is aligned to horizontal as illustrated, the second portion is at angle to horizontal that is preferably 20 degrees or approximately 20 degrees.

Further, it is to be appreciated that while not specifically illustrated, the first and second portions 48, 52 may continuously taper in thickness along their length from the proximal end 32 to the distal end 30 of the rod 22, such that a thickness of the rod 22 proximate the distal end 30 is less than or more than a thickness of the rod 22 proximate the proximal end 32. Moreover, the rod 22 can have any of the cross-sectional configurations described herein with reference to the first elongated member 24, but it is preferable that the rod has a circular cross-section so as to avoid damage to oropharyngeal tissue during use.

The bougie 20 further includes a first hole 21 extending through a sidewall of the third elongated element 36 for receiving a tip of a locking mechanism extending through a sidewall of the second elongated element 34, as described with reference to FIG. 2. Moreover, the bougie 20 includes a second hole 23 extending through a sidewall of the second elongated element 34 for receiving a tip of a locking mechanism extending through a sidewall of the first elongated element 24, as described herein.

In the illustrated implementation in FIG. 1, the bougie or stylet 20 has a length from the terminal end of the tip 46 to the proximal end 44 of the third elongated member 36 of between 310 and 330 millimeters (“mm”) and more preferably, of 318.5 mm or approximately 318.5 mm (i.e. between 317 and 320 mm) in the collapsed position illustrated in FIG. 1. The rod 22 has a maximum outer dimension (i.e. a diameter, in an implementation) of between 1 and 3 mm, or more preferably, 2 mm or approximately 2 mm (i.e. between 1.8 and 2.2 mm). The first portion 52 of the rod 22 preferably has a length of between 14 and 16 mm or more preferably, 15.43 mm or approximately 15.43 mm (i.e. between 14 and 17 mm). The second portion 48 of the rod 22, including the tip 46, has a length of between 35 and 45 mm, or more preferably, 40 mm or approximately 40 mm (i.e. between 39 and 41 mm). The tip 46 preferably has a maximum outer dimension (i.e. a diameter, in an implementation) of between 3 and 6 mm, or more preferably, 4.5 mm or approximately 4.5 mm (i.e. between 4 and 5 mm).

FIG. 2 illustrates the bougie 20 is an extended configuration. Notably, as indicated above, FIG. 2 is not necessarily drawn to scale in order to avoid obscuring features of the implementation. As shown in FIG. 2, the first tubular or elongated element 24 includes an internal longitudinal bore 54, the second tubular or elongated element 34 includes an internal longitudinal bore 56 and the third tubular or elongated element 34 includes an internal longitudinal bore 58. The longitudinal bore 54 of the first element 24 is sized and shape to receive the rod 22, which is preferably solid (i.e. does not have a longitudinal bore). As described herein, the rod 22 is preferably permanently fixed in place in the longitudinal bore 54, but may be slidably or telescopically received within the longitudinal bore 54 of the first element 24 in other implementations. In an implementation where the rod 22 is permanently fixed in the longitudinal bore 54 of the first element 24, the space between the rod 22 and the longitudinal bore 54 may further be filled with a dense material in order to increase strength and reduce the bending capability of the first elongated element 24. Such dense material may include various polymers or glues that are hardened in place or wood, metal, or plastic, for example.

The longitudinal bore 56 of the second elongated element 34 is sized and shaped to slidably or telescopically receive the first elongated element 24 and the longitudinal bore 58 of the third elongated element 36 is sized and shaped to slidably or telescopically receive the second elongated element 56. As such, it is preferable that each of the longitudinal bores 56, 58 remain hollow (i.e. without any material present in the bores 56, 58), but in other implementations, the bougie 20 is manufactured in the extended configuration shown in FIG. 2 with each of the bores 54, 56, 58 filled with dense material, as above.

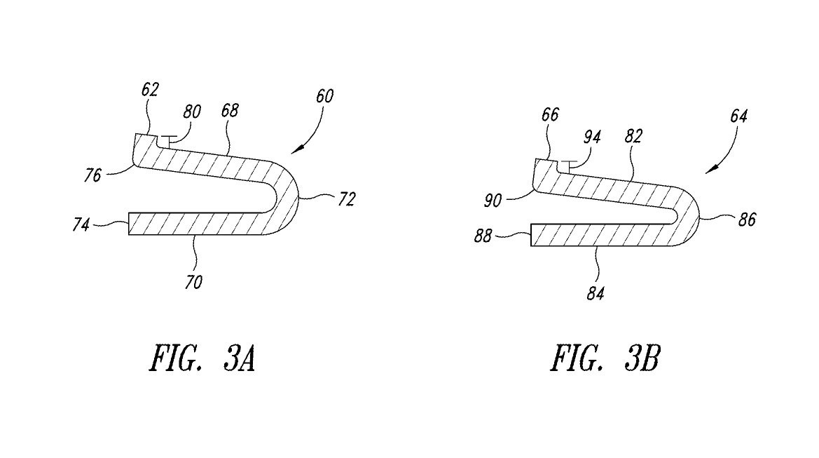

FIG. 2 further illustrates a locking mechanism 60 that permanently couples the third tubular or elongated member 36 to the second tubular or elongated member 34 when the bougie 20 is in the extended position or configuration, as illustrated. While the locking mechanism 60 will be described further with reference to FIG. 3A, FIG. 2 illustrates that the locking mechanism 60 has a tip or pin 62 that preferably extends through a hole in a sidewall of the second elongated element 34 proximate the proximal end 40 of the second elongated element 34. When the second member 34 is slidably extended from the third elongated element 36 to the extended configuration shown in FIG. 2, the tip 62 aligns with the hole 21 in the sidewall of the third member 36 and extends into the hole 21 to lock the second member 34 in the extended configuration. In other words, in the extended configuration, the tip 62 extends through a hole in the sidewall of the second elongated element 34. In an implementation, a terminal end of the tip 62 aligns with or is planar to, an outermost surface of the sidewall of the third elongated element 36, while in other implementations, the terminal end of the tip 62 extends beyond the outermost surface of the third elongated element 36, wherein the outermost surface may be flat and planar as illustrated in FIG. 2, or may be rounded, as described herein.

Moreover, the bougie 20 preferably includes a locking mechanism 64 that permanently couples the second elongated element 34 to the first elongated element 24 when the bougie 20 is in the extended configuration, as illustrated in FIG. 2. While the locking mechanism 64 will be described further with reference to FIG. 3B, FIG. 2 illustrates that the locking mechanism 64 has a tip or pin 66 that preferably extends through a hole in a sidewall of the first element 24. When the first member 24 is slidably extended from the second elongated element 34 to the extended configuration shown in FIG. 2, the tip 66 aligns with the hole 23 in the sidewall of the second member 34 and extends into the hole 23 to lock the first element 24 in the extended configuration. In other words, the tip 66 extends through both a hole in the sidewall of the first elongated element 24 and the hole 23 in the sidewall of the second elongated element 34. In an implementation, a terminal end of the tip 66 aligns with or is planar to, an outermost surface of the sidewall of the second elongated element 34, while in other implementations, the terminal end of the tip 66 extends beyond the outermost surface of the second elongated element 34. Moreover, the outermost surface may be flat and planar, as illustrated in FIG. 2, or may be rounded, as described herein.

In the illustrated implementation, the terminal end of the tip 62 of locking mechanism 60 is positioned further from a central axis extending through each of the elongated members 24, 34, 36 than the terminal end of the tip 66 of locking mechanism 64. Although not specifically illustrated, in implementations where the rod 22 is slidably received in the first elongated element 24, an additional locking mechanism, which may be similar in function to locking mechanisms 60, 64 may be present to lock or permanently hold the rod 22 in the extended configuration within the first elongated element 24. In the context of FIG. 2, “lock” or “permanently hold” are construed to include “cannot be collapsed,” which corresponds to the locking mechanisms 60, 64 being able to withstand applied axial forces (i.e. compression or tension) at least greater than the amount of force exerted on the device 20 by an average person (i.e. a force of at least 100 pounds, or more preferably, of at least 200 or more pounds) without allowing the elements 24, 34, 36 or the rod 22, as the case may be, to return to the collapsed configuration or be forced to separate into component pieces from the extended configuration. In any event, the locking mechanisms 60, 64 are designed to withstand the maximum potential force exerted against the bougie 20 and locking mechanisms 60, 64 when the bougie 20 is inserted into an endotracheal passageway, or constricted airway, of a patient, as described herein. It is to be appreciated that the description contained herein with respect to an amount of force includes the equivalent pressure applied to an end surface of the device according to the equation P=F/A, where P is pressure, F is force, and A is area of the surface the force is applied to.

In the extended configuration shown in FIG. 2, the bougie 20 includes the third elongated element 36 having a length of between 240 and 270 mm, or more preferably, 259.5 mm or approximately 259.5 mm (i.e. between 258 and 261 mm). The second elongated element 34 has a length of between 240 and 270 mm, but more preferably, 255 mm or approximately 255 mm (i.e. between 254 and 256 mm). As such, an overlap 25 between the third elongated element 36 and the second elongated element 34 in the elongated configuration shown in FIG. 2 is between 65 and 75 mm, but more preferably is 70 mm or approximately 70 mm (i.e. between 69 and 71 mm). In other words, in the elongated or extended configuration, approximately 70 mm of the second elongated element 34 are contained within the longitudinal bore 58 of the third elongated element 36.

The first elongated element 24 has a length of between 230 and 250 mm, but more preferably, is 242 mm or approximately 242 mm (i.e. between 241 and 243 mm). An overlap 27 between the second elongated element 34 and the first elongated element 24 in the elongated configuration shown in FIG. 2 is between 70 and 80 mm, but more preferably is 76.5 mm or approximately 76.5 mm (i.e. between 75 and 78 mm). In other words, in the elongated configuration, approximately 76.5 mm of the first elongated element 24 are contained within the longitudinal bore 56 of the second elongated element 34. As such, a length of the overlap 27 is preferably greater than a length of the overlap 25, although in other implementations, the lengths of the overlaps 25, 27 are the same, or the length of the overlap 27 is less than the length of the overlap 25. In other words, in an implementation, the portion of the second elongated element 34 contained within the longitudinal bore 58 of the third elongated element 36 is less than the portion of the first elongated element 24 contained within the longitudinal bore 56 of the second elongated element 34, although in other implementations, the portions may be the same, or the portion of the second elongated element 34 contained within the longitudinal bore 58 of the third elongated element is greater than the portion of the first elongated element 24 contained within the longitudinal bore 56 of the second elongated element 34.

Moreover, in the extended configuration, approximately 165.5 mm (i.e. between 164 and 167 mm) of the first elongated element 24 extend from the second elongated element 34 and similarly, approximately 185 mm (i.e. between 184 mm and 186 mm) of the second elongated element 34 extend from the third elongated element 36. A total length of the elongated members 24, 34, 36 in the extended configuration is between 600 mm and 620 mm, but more preferably is equal to 610 mm or approximately 610 mm (i.e. between 605 and 615 mm). Combined with the length of the rod 22, the overall length of the bougie 20 in the extended configuration is between 650 and 680 mm, but more preferably is 665.43 mm or approximately 665.43 mm (i.e. between 663 and 668 mm).

FIG. 3A illustrates the locking mechanism 60 in additional detail. The locking mechanism 60 includes a first arm 68 and a second arm 70 connected by a transition portion 72. The arms 68, 70 are preferably straight and elongated, with the transition portion 72 comprising a curved, semi-circular section with an angle of rotation around an outermost edge of the transition portion 72 being equal to 173 degrees, or approximately 173 degrees (i.e. between 170 and 175 degrees) and a diameter of the outermost edge being equal to 3.25 mm or approximately 3.25 mm (i.e. between 2.5 mm and 4 mm). As such, an angle 78 between the arms 68, 70 is preferably 7 degrees or approximately 7 degrees (i.e. between 6 and 8 degrees). The second arm 70 terminates in a terminal end 74 that is preferably flat and planar. The first arm 68 terminates with pin 62, which preferably has a height 80 of approximately 0.55 mm (i.e. between 0.5 and 0.6 mm) relative to first arm 68.

The pin 62 is connected to the first arm 68 by a curved portion 76. The locking mechanism 60 preferably has a maximum thickness (i.e. a diameter, in an implementation) equal to 1 mm, or approximately 1 mm (i.e. between 0.5 mm and 1.5 mm) over an entire length of the locking mechanism 60. Each arm 68, 70 is preferably 7.3 mm in length or approximately 7.3 mm in length (i.e. between 7 and 8 mm). In other words, a length from the terminal end 74 of the second arm 70 to an outermost edge of the transition portion 72 is equal to 7.3 mm or approximately 7.3 mm and similarly, a length of the first arm 68 from an outermost edge of the curved section 76 to the outermost edge of the transition portion 72 is preferably equal to 7.3 mm or approximately 7.3 mm. In addition, the locking mechanism 60 can have any of the cross-sectional configurations discussed above with reference to the first elongated member 24, but in an implementation, the cross-sectional configuration is preferably a circle with a constant diameter over an entire length of the locking mechanism 60. In yet other implementations, the thickness or diameter of the locking mechanism 60 tapers along the entire length, including along the height 80 of the pin 62, along either of the arms 68, 70, or along the transition portion 72, such that a thickness or maximum outer dimension in one portion of the locking mechanism 60 is greater than or less than a thickness in other portions.

FIG. 3B illustrates the locking mechanism 64 in additional detail. The locking mechanism 64 includes a first arm 82 and a second arm 84 connected by a transition portion 86. The arms 82, 84 are preferably straight and elongated, with the transition portion 86 comprising a curved, semi-circular section with an angle of rotation around an outermost edge of the transition portion 86 being equal to 173 degrees, or approximately 173 degrees (i.e. between 170 and 175 degrees) and a diameter of the outermost edge being equal to 2.55 mm or approximately 2.55 mm (i.e. between 2 mm and 3 mm). As such, an angle 92 between the arms 82, 84 is approximately 7 degrees (i.e. between 6 and 8 degrees). The second arm 84 terminates in a terminal end 88 that is preferably flat and planar. The first arm 82 terminates with pin 66, which preferably has a height 94 of approximately 0.55 mm (i.e. between 0.5 and 0.6 mm) relative to the first arm 82.

The pin 66 is connected to the first arm 82 by a curved portion 90. The locking mechanism 64 preferably has a maximum thickness (i.e. a diameter, in an implementation) equal to 1 mm, or approximately 1 mm (i.e. between 0.5 mm and 1.5 mm) over an entire length of the locking mechanism 64. Each arm 82, 84 is preferably 7.3 mm in length or approximately 7.3 mm in length (i.e. between 7 and 8 mm). In other words, a length from the terminal end 88 of the second arm 84 to an outermost edge of the transition portion 86 is equal to 7.3 mm or approximately 7.3 mm and similarly, a length of the first arm 82 from an outermost edge of the curved section 90 to the outermost edge of the transition portion 86 is preferably equal to 7.3 mm or approximately 7.3 mm. In addition, the locking mechanism 64 can have any of the cross-sectional configurations discussed above with reference to the first elongated member 24, but in an implementation, the cross-sectional configuration is preferably a circle with a constant diameter over an entire length of the locking mechanism 64. In yet other implementations, the thickness or diameter of the locking mechanism 64 tapers along the entire length, including along the height 94 of the pin 66, along either of the arms 82, 84, or along the transition portion 86, such that a thickness or maximum outer dimension in one portion of the locking mechanism 64 is greater than or less than a thickness in other portions.

It is to be appreciated that the discussion contained herein regarding the size or dimensions of certain features or implementations of the present disclosure includes dimensions which are more or less than those described and as such, implementations of the present disclosure are not to be construed to be limited by these dimensions. Similarly, the discussion contained herein with respect to various angles between features of implementations of the present disclosure includes angles which are more or less than those described and as such, implementations of the present disclosure are not limited by such angles. It is to be further appreciated that any description of dimensions in terms of certain units are to be construed as including equivalent dimensions in other units according to standard conversion equations between units of measure.

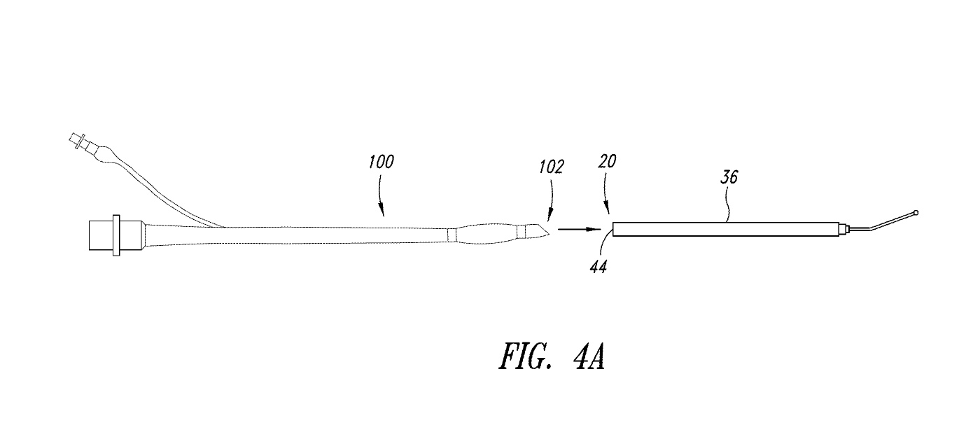

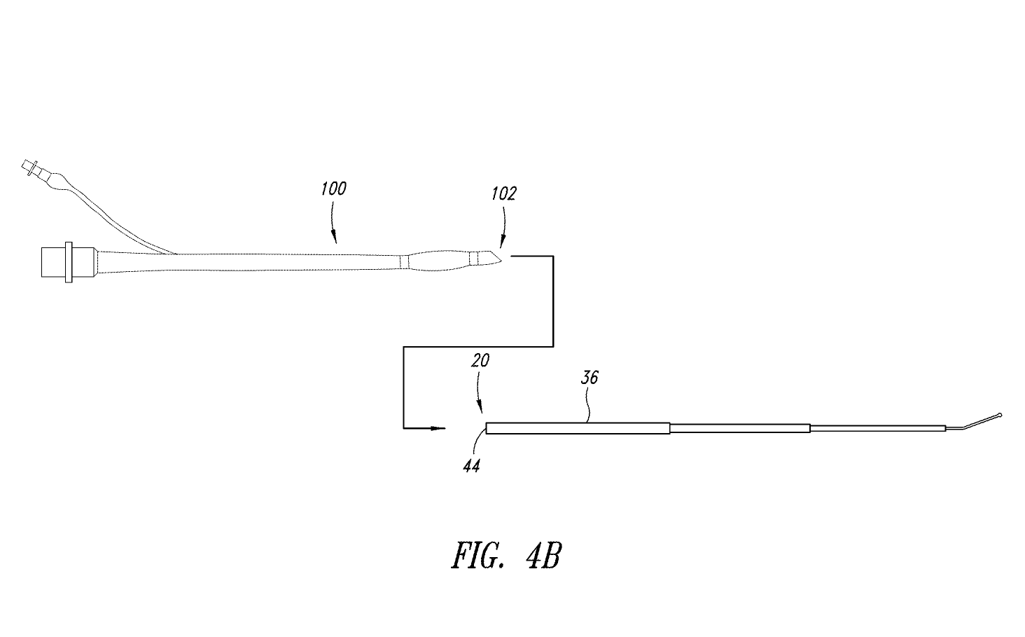

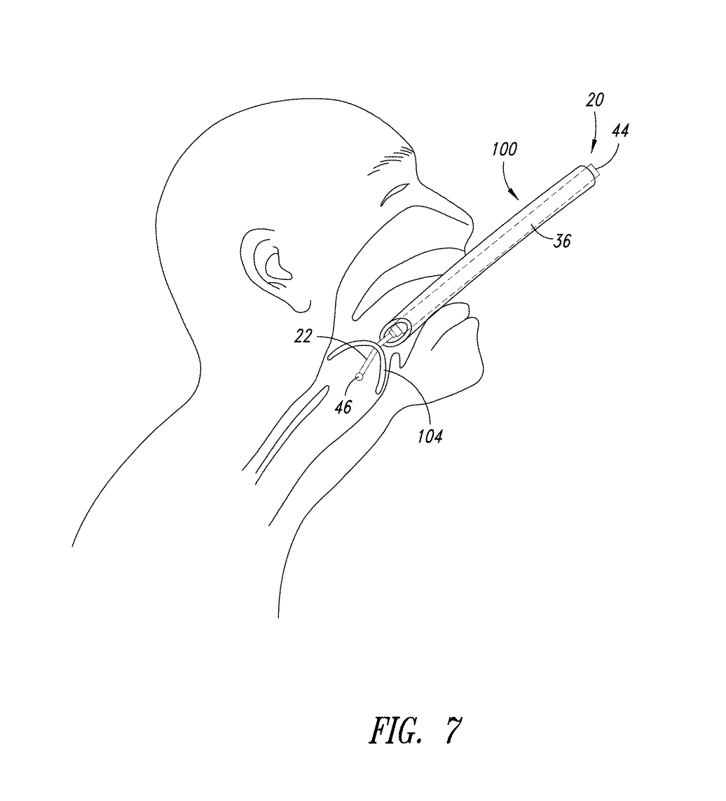

FIG. 4A illustrates a representative use condition of the bougie as a stylet 20, which will be described more fully with reference to FIG. 7. In operation, a user, such as a medical care provider, carries stylet 20 in their scrub pocket or medical tool kit, for example. Prior to use, the stylet 20 may be sterilized and placed in a sterile package. When the user encounters a patient who is suffering from a constricted airway, and in particular, an adolescent patient, the user then removes the stylet 20 from the scrub pocket or medical took kit and removes the packaging, as necessary. Then, the stylet 20 can be arranged such that the proximal end 44 of the third elongated element 36 can be inserted into an opening 102 at a terminal end of the endotracheal intubation tube 100 as shown in FIG. 4A.

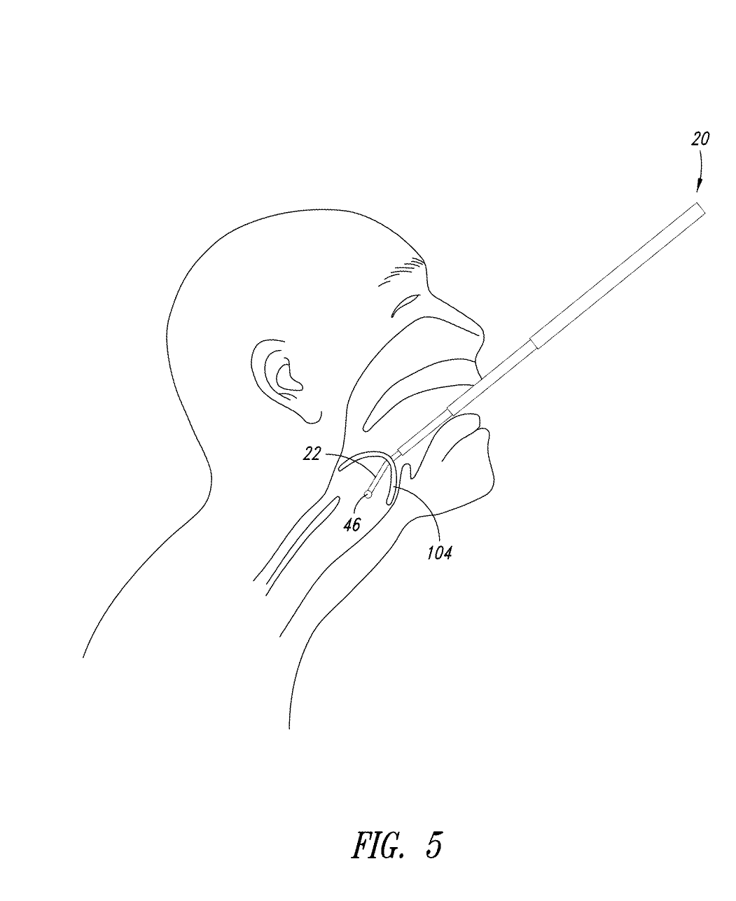

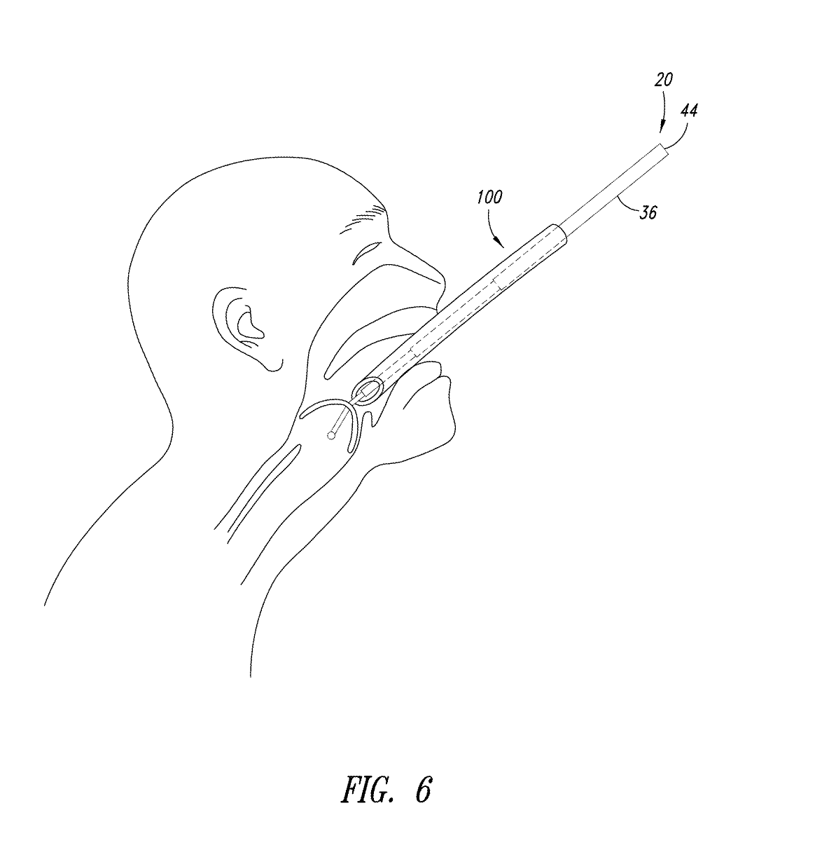

FIG. 4B illustrates an alternative representative use condition of the bougie 20, which will be described more fully with reference to FIGS. 5-6. In operation, a user, such as a medical care provider, carries bougie 20 in the collapsed position in their scrub pocket or medical tool kit, for example. Prior to use, the bougie 20 may be sterilized and placed in a sterile package. When the user encounters a patient who is suffering from a constricted airway, user then removes the bougie 20 from the scrub pocket or medical took kit and removes the packaging, as necessary, and extends the bougie to the extended configuration. Then, the stylet 20 can be arranged such that the proximal end 44 of the third elongated element 36 can be inserted into the opening 102 at the terminal end of the endotracheal intubation tube 100 as shown in FIG. 4B.

FIGS. 5-6 elaborate on the use condition described above with reference to FIG. 4B. In operation, once the bougie 20 is extended to the extended configuration, such that the locking mechanisms 60, 64 lock the bougie 20 in the extended configuration, the user inspects the oropharynx of the patient using a laryngoscope (not shown) and the vocal cords are identified. The rod 22 and the tip 46 are gently advanced past the lips of the patient and advanced just distal to the vocal cords 104, as illustrated in FIG. 5. Then, an assistant advances the endotracheal intubation tube 100 over the proximal end 44 of the third elongated member 36 and the tube 100 is advanced over the bougie 20 to the user's fingers, which are securely grasping the bougie 20. At this time the user releases the bougie 20 and allows the endotracheal tube 100 to slide past their fingers. The user then grasps the endotracheal tube 100 and advances the endotracheal tube 100 over the bougie 20 and gently passes the endotracheal tube just distal to the vocal cords 104. Once the endotracheal tube cuff is visualized past the vocal cords 104, the user continues to securely grasp the endotracheal tube 100 while the assistant removes the bougie 20 by grasping the bougie 20 and pulling back on the axis of the bougie 20 until the bougie 20 is no longer in the endotracheal tube 100. Finally, the endotracheal tube cuff is inflated and then connected to a resuscitation bag and the patient is ventilated.

Additionally or alternatively, in the event of an emergency where an assistant is not able to assist with intubation, the bougie 20 can be used with the endotracheal tube 100 loaded onto the bougie 20 prior to inspection of the oropharynx with the laryngoscope, such that the user or operator can advance the tube 100 over the bougie 20 without assistance.

FIG. 7 illustrates a similar use condition for the bougie as the stylet 20 and further elaborates on the arrangement in FIG. 4A. Importantly, this configuration is particularly well suited to adolescents, children, or individuals with a smaller airway that require a smaller endotracheal tube 100. Once the stylet 20 is removed from the packaging, the user inspects the oropharynx of the patient using a laryngoscope (not shown) and the vocal cords are identified. The rod 22 and the tip 46 are gently advanced past the lips of the patient and advanced just distal to the vocal cords 104, as illustrated in FIG. 7. Then, an assistant advances the endotracheal intubation tube 100 over the proximal end 44 of the third elongated member 36 and the tube 100 is advanced over the stylet 20 to the user's fingers, which are securely grasping the stylet 20. At this time the user releases the stylet 20 and allows the endotracheal tube 100 to slide past their fingers. The user then grasps the endotracheal tube 100 and advances the endotracheal tube 100 over the stylet 20 and gently passes the endotracheal tube just distal to the vocal cords 104. Once the endotracheal tube cuff is visualized past the vocal cords 104, the user releases their grasp and the assistant removes the stylet 20 by grasping the stylet 20 and pulling back on the axis of the stylet 20 until the stylet 20 is no longer in the endotracheal tube 100. Finally, the endotracheal tube cuff is inflated and then connected to a resuscitation bag and the patient is ventilated.

Further, it is to be appreciated that particularly with respect to use of the stylet 20 with a child, which requires use of a small endotracheal tube, the stylet 20 may not include the third elongated element 36, but rather, may only include the first and second elongated elements 24, 34 in order to reduce the outer diameter of the stylet 20 to accommodate the smaller endotracheal intubation tube 100. In such an implementation, the first elongated element 24 may still be extendable to the extended configuration relative to the second elongated element 34, such that the reduced size stylet 20 can also be used as a smaller sized bougie 20 according to the methods of use described herein.

The bougie 20 may also be used as a stylet 20 with adult patients. In this stylet configuration, which corresponds to the collapsed position or configuration, the endotracheal tube 100 is loaded onto the stylet 20 and then the stylet 20 and tube 100 are advanced as one unit past vocal cords 104, wherein the user or operator continues to securely grasp the endotracheal tube 100 while the assistant removes the stylet 20, as described herein. In some cases, it may be preferable to start with the bougie 20 as a stylet (i.e. collapsed) for all patients, and then extend the bougie 20 to the extended configuration or position, if needed based on the difficulty to intubate the patient.

As will be readily appreciated from the foregoing, this new bougie, such as bougie 20, is telescoping so that can fit into a provider's scrub pocket for immediate access in an airway emergency. It is made of hard, preferably unbendable material, telescopes to an approximate length of two feet or more than two feet, and has an angled tip. The distal tip is round and smooth to allow the device to slide along the oropharyngeal tissue without tearing of the tissue. An endotracheal tube can be advanced over this telescoping bougie for placement in the trachea. Because of its small size, it can be stored in a myriad of places and easily accessed immediately by emergency personnel in the field, emergency department, intensive care unit, and in any location during a code blue situation.

This new telescoping intubation bougie can be sterilized and easily carried by critical care providers and EMS personnel, making it very quick to facilitate airway access in those patients with a difficult airway, thus saving precious time and patient lives. Video technology can be added to allow direct visualization of the airway via a device at the distal tip.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A device, comprising:
a first elongated element having a distal end and a proximal end, and a rigid body with a longitudinal axial bore that is formed of non-malleable, unbendable material;
a second elongated element having a distal end and a proximal end, and a rigid body with a longitudinal axial bore that is formed of non-malleable, unbendable material, the first elongated element sized and shaped to be received in the longitudinal axial bore of the second elongated element and to move within the longitudinal axial bore between a collapsed position in which the first elongated element is slidably received within the longitudinal axial bore of the second elongated element and an extended position in which the first elongated element slidably extends out of the second elongated element;
a locking mechanism that holds the first elongated element in the extended position within the second elongated element;
a rigid rod having a distal end and a proximal end, the rod having a body formed of non-malleable, unbendable material and sized and shaped to be received within the longitudinal axial bore of the first rigid elongated element; and
a rigid tip formed on the distal end of the rod, the tip having a smooth, spherical terminal end and structured to project out of the first elongated element in either the collapsed or the extending position.

2. The device of claim 1 wherein the first rigid elongated element has a geometric cross-sectional configuration of one from among a square, a triangle, a rectangle, a circle, an oval, and a diamond.

3. The device of claim 1 wherein the locking mechanism permanently locks the first elongated element in the extended position so that it cannot be returned to the collapsed position.

4. The device of claim 1, further comprising a third elongated element formed of non-malleable, unbendable material and structured to slidably receive the second elongated element.

5. The device of claim 4 further comprising a sleeve between the rod and the longitudinal axial bore of the first elongated member, the sleeve, the rod, and the longitudinal axial bore of the first elongated member permanently coupled in a crimp fit.

6. The device of claim 4, further comprising a second locking mechanism structured to permanently lock the second elongated element in an extended position relative to the third elongated element.

7. The device of claim 1 wherein a portion of the rigid rod proximate the rigid tip is at an angle to horizontal that is approximately 20 degrees.

8. A device, comprising:
a first elongated element having an inflexible body between opposing distal ends and a longitudinal axial bore extending through the body between the opposing terminal ends;
an inflexible rod received in the longitudinal axial bore of the first elongated element and coupled to the longitudinal axial bore of the first elongated element, the inflexible rod having a first portion proximate the first elongated element and a second portion separated from the first portion by a transition portion, wherein the first portion is at angle to the second portion; and
a rigid tip coupled to and extending from the second portion, the tip having a smooth, spherical terminal end spaced from a distal end of the first elongated element.

9. The device of claim 8 wherein the angle is between 10 and 30 degrees.

10. The device of claim 8 wherein the angle is approximately 20 degrees.

11. The device of claim 8 wherein the first elongated element has a geometric cross-sectional configuration of one from among a square, a triangle, a rectangle, a circle, an oval, and a diamond.

12. The device of claim 8 further comprising:
a second elongated element having a longitudinal bore, the first elongated element telescopically received in the longitudinal bore of the second elongated element, the first elongated element slidable along the longitudinal bore of the second elongated element between a collapsed configuration and an extended configuration.

13. The device of claim 12 further comprising:
a locking mechanism structured to permanently lock the first elongated member in the extended configuration.

14. The device of claim 12 further comprising:
a third elongated element with a longitudinal bore, the second elongated element telescopically received in the longitudinal bore of the third elongated element, wherein the second elongated element is slidable along the longitudinal bore of the third elongated element between a collapsed configuration and an elongated configuration.

15. The device of claim 14 further comprising:
a locking mechanism structured to permanently lock the second elongated member in the extended configuration.
